(19) United States
(12) Patent Application Publication
(10) Pub. No.: US 2026/0198774 A1
(43) Pub. Date: Jul. 16, 2026

(54) SYSTEM FOR CARRYING OUT A CONFOCAL OPHTHALMOSCOPY

(71) Applicant: Heidelberg Engineering GmbH, Heidelberg (DE)

(72) Inventor: Christoph BROSCHE, Eppelheim (DE)

(73) Assignee: Heidelberg Engineering GmbH, Heidelberg (DE)

(21) Appl. No.: 19/131,995

(22) PCT Filed: Sep. 7, 2023
(86) PCT No.: PCT/EP2023/074559

§ 371 Date: May 22, 2025

(30) Foreign Application Priority Data
Nov. 22, 2022 (DE) 10 2022 130 879.0

(51) Int. Cl. A61B 3/12; A61B 3/00; A61B 3/10

(52) U.S. Cl. CPC A61B 3/1225; A61B 3/0025; A61B 3/102

(57) ABSTRACT

The invention relates to a system for carrying out a confocal ophthalmoscopy, comprising an illumination device for illuminating a sample (13), a lens assembly with multiple lenses (6-11) for directing light beams from the illumination device to the sample (13) and back from the sample to the detector (3), wherein the objective of the invention is to provide a system permitting imaging with the best possible image quality and the invention is thus characterised in that the illumination device has a DMD chip (2) with which a light beam can be directed to the sample (13) and with which a light beam reflected back from the sample (13) can be directed to the detector (3).

The invention relates to a system according to the preamble of claim 1.

Lasers are used in laser scanning ophthalmoscopy (SLO). A laser is guided over the ocular fundus to examine it and emits light onto the ocular fundus. The ocular fundus reflects light and this reflected light is analyzed and used to obtain an image. The ocular fundus is typically scanned by the laser, wherein a grid pattern is generated. The grid pattern typically extends from left to right and then vertically. In this way, it is possible to clearly image a zone of the ocular fundus to be examined.

Laser scanning ophthalmoscopy is based on a similar principle as laser scanning microscopy. In laser scanning ophthalmoscopy, in contrast to laser scanning microscopy, the lens of the human eye is used as the objective lens. In laser scanning microscopy, an artificial objective lens is provided in a microscope.

Classic scanning technologies are often used for optical imaging in ophthalmology. These include, among others, line scan systems or confocal scanning laser systems having two separate scanners for the X direction and the Y direction.

Devices for carrying out a laser scanning ophthalmoscopy using classic scanners generally include relatively expensive components, such as a line camera or oscillating scanner. Conventional line cameras are less light-sensitive in comparison to individual detectors and the imaging is only confocal in one axis if they are used. Oscillating resonant scanners can shift, have a sinusoidal movement, and can sometimes develop high volume levels.

The invention is therefore based on the object of specifying a system, using which imaging is possible with the best possible image quality.

The present invention achieves the above-mentioned object by way of the features of claim 1.

Accordingly, the illumination apparatus has a DMD chip, by which a light beam can be guided to a sample and by which a light beam reflected from the sample to it can be guided to a detector.

It has first been recognized according to the invention that imaging systems which use so-called digital mirror devices (DMD) as scanners are already known. It has then been recognized that in the known concepts, DMDs or DMD chips are only used as an illumination unit.

Furthermore, it has been recognized that the light returning from the eye, thus reflected back or scattered, does not run via the DMD chip again in the prior art, but rather is deflected beforehand using beam splitters onto a high-resolution 2D camera chip, which then generates an image.

Furthermore, it has been recognized that this structure is not confocal, due to which the image quality is reduced. Conventional CMOS and CCD camera chips having high resolution are significantly less light-sensitive in comparison to individual detectors.

It has also been recognized that the image rate for high-resolution pictures is relatively slow if the entire DMD chip is scanned pixel by pixel.

Finally, it has been recognized according to the invention that high image rates having high resolution are implementable by a confocal structure using DMD chips. Such a system is advantageously distinguished by a good image quality due to confocal structure. The system advantageously has essentially no large moving parts, is absolutely optically stable, and is silent.

The use of DMD chips ensures the use of a long-lived robust technology. Robust digital imaging, in which no scanner drifts, sinusoidal curves, jitter, or the like occurs, advantageously takes place using the system.

A DMD chip displays an occupancy with optical individual elements, namely very small mirrors or also micromirrors. In order to guide or reflect light incident from one direction on a mirror in another direction, each individual mirror is movable, in particular tiltable, independently from the others.

Against this background, a light beam or light beam bundle could be guided on the same optical path from the DMD chip to the sample and from this sample back to the DMD chip as a reflected light beam or reflected light beam bundle so that the same individual mirror of the DMD chip is usable for illuminating the sample using the light beam or light beam bundle incident thereon and for receiving the light beam or light beam bundle reflected from the sample. The system thus permits the implementation of a variable pinhole size and pinhole shape by interconnecting individual mirrors of the DMD chip. Depending on the resolution, the system is cost-effective and compact and very inexpensive in comparison to alternative scanning systems.

The detector could be designed here as a surface detector and/or MPPC array detector. In this way, a confocal structure having a DMD chip and digital scanning are enabled. On the object side, the DMD chip is imaged on the retina if the human eye is used as the sample, and, on the detector side, the DMD chip is imaged on a detector array.

The DMD chip could have a surface which is divided into a specific number of segments, wherein the detector has a surface which is also divided into a specific number of segments, and wherein the segments of the DMD chip each optically correspond with the segments of the detector in order to generate an image. The DMD chip is thus optically imaged on the detector array.

All segments of the DMD chip could be equal in size, wherein all segments of the detector are equal in size. The DMD chip is configurable due to the large number of mirrors such that it simulates individual segments, in particular their size, whereas the detector has real spatially-physically bounded segments. The DMD chip is therefore adjustable to the detector. Alternatively or additionally, the segments of the DMD chip could be the same size as the segments of the detector. The division of the DMD chip and the detector into individual identical segments permits them to be scanned in parallel.

Each segment of the DMD chip could be assigned precisely one segment of the detector, so that the DMD chip can be imaged or projected on the sample side on the surface of the sample to be examined and on the detector side on the surface of the detector. The light returning from the sample or the eye can thus run via the DMD chip and can be guided thereby onto the segmented detector or a segmented detector array. The DMD chip is not only used for the illumination in this case. The individual mirrors of the DMD chip function as a pinhole, thus actually have a confocal effect. The pinhole size and the pinhole shape are therefore variable.

The illumination apparatus could comprise an illumination unit, wherein the light coming from the illumination unit can be guided onto the DMD chip. The DMD chip can thus be used for the illumination and is part of the illumination apparatus. Alternatively, the light coming from the illumination unit can be guided by means of a TIR prism onto the DMD chip. A TIR prism generally consists of two prisms laminated on one another, which guide incident light particularly compactly onto a DMD chip.

A beam splitter, by which a light beam reflected from the sample can be deflected onto the detector, could be arranged between the illumination unit and the DMD chip and/or a TIR prism. Reflected light thus strikes the detector and the confocal principle is maintained.

A light beam originating from the illumination unit could be able to be guided by a first lens and a second lens before the light beam strikes the TIR prism and/or the DMD chip. In this way, it is possible to place a beam splitter between the two mentioned lenses, which are axially spaced apart from one another.

Against this background, a beam splitter could be arranged between a first lens and a second lens. Reflected light can be guided to the detector by this beam splitter.

Outgoing light beams from the DMD chip to the sample could be able to be guided by an excitation lens or third lens alone or optionally furthermore by a fourth lens and a fifth lens before they strike the sample. A confocal structure is implementable by each of the two lens configurations, in which the lens of the human eye functions as part of an optical arrangement. In one case, only a single lens is used to produce a structure with few parts, and in the other variant, three lenses are used for, for example, simple housing of additional optical components.

A detector lens or sixth lens could be arranged between a beam splitter and the detector. Light can thus be focused on the detector. The sample or the human eye is imaged on the DMD chip and the detector.

An individual mirror of the DMD chip could function as a pinhole aperture. Each individual mirror of the DMD chip thus functions as a so-called pinhole. The confocal principle is thus implemented.

In an arrangement which comprises a system of the type described here, the illumination apparatus could comprise an illumination unit or swept source OCT illumination unit, the emitted light of which can be split into a sample arm and a reference arm, wherein the light signals from sample arm and reference arm can be overlaid at the detector such that they interfere, and wherein OCT images can be generated from the interference signals by means of an evaluation apparatus.

A DMD chip is thus used for the OCT imaging. The system used is distinguished by fixed imaging.

The light of the sample arm could be able to be guided via a feed lens to the DMD chip, wherein the light of the reference arm can be guided via a deflection mirror or reference light beam splitter to the DMD chip. If a swept source OCT illumination unit is used, it illuminates the DMD chip.

The system described here can be used both for confocal laser scanning 2D pictures and also for OCT imaging. The system described here can in particular also be combined with swept source OCT (SS-OCT).

The designation optical coherence tomography (typically abbreviated to OCT) is understood as an imaging method. Two-dimensional and three-dimensional images can be obtained from light scattering structures using this method.

The imaging of the retina of the human eye is described in this description, but the described system can also be used for other applications.

### IN THE FIGURES

FIG. 1 shows a system for carrying out a confocal ophthalmoscopy having multiple lenses between the DMD chip or a TIR prism and the human eye,

FIG. 2 schematically shows, on the left, a detector having a surface divided into segments, in the center a DMD chip having a surface divided into simulated segments, wherein the size and number of the segments correspond to those of the detector, and on the right an image captured using the DMD chip and the detector, which is composed of segments,

FIG. 3 shows a system for carrying out a confocal ophthalmoscopy having only one lens between the DMD chip or a TIR prism and the human eye, and

FIG. 4 shows an arrangement for OCT imaging.

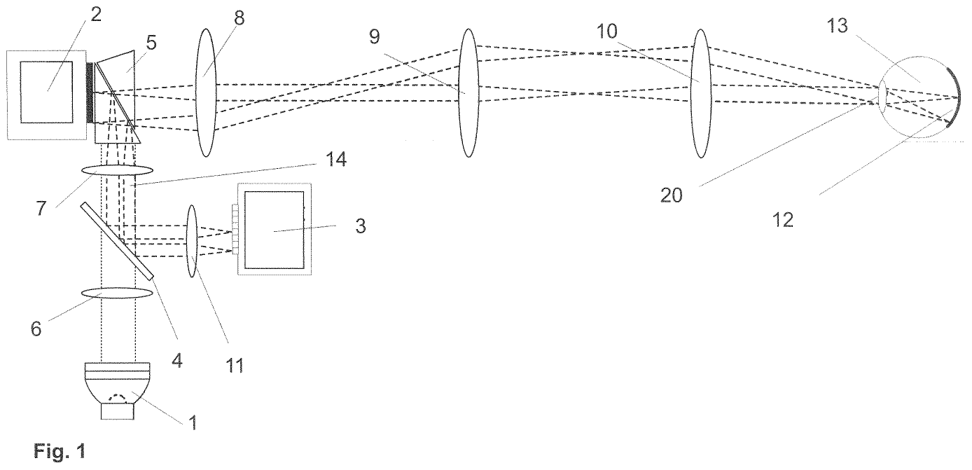

FIG. 1 shows a system for carrying out a confocal ophthalmoscopy, comprising an illumination apparatus for illuminating a sample 13, a lens arrangement having multiple lenses 6-11 for guiding light beams from the illumination apparatus to the sample 13 and from this sample back to a detector 3. The illumination apparatus comprises a DMD chip 2, by which a light beam can be guided to the sample 13 and by which a light beam reflected back from the sample 13 to it can be guided to the detector 3.

FIG. 1 schematically shows in this respect the function of a confocal optical arrangement for imaging the ocular fundus, in particular the retina 12. The human eye is used as the sample 13. The arrangement comprises the system having an illumination apparatus, which comprises an illumination unit 1 and the DMD chip 2.

The system furthermore comprises the detector 3, which is designed as an MPPC array detector, wherein MPPC stands for “multi-pixel photon counter”, and various further optical components for beam guiding, namely a beam splitter 4, a TIR prism 5, and various optical lenses 6 to 11.

A light beam or light beam bundle is guided on the same optical path from the DMD chip 2 to the sample 13 and from this back to the DMD chip 2 as a reflected light beam or reflected light beam bundle, so that the same individual mirror of the DMD chip 2 is usable for illuminating the sample 13 using the light beam or light beam bundle incident thereon and for receiving the light beam or light beam bundle reflected from the sample 13.

The detector 3 is designed as a surface detector, namely as an MPPC array detector.

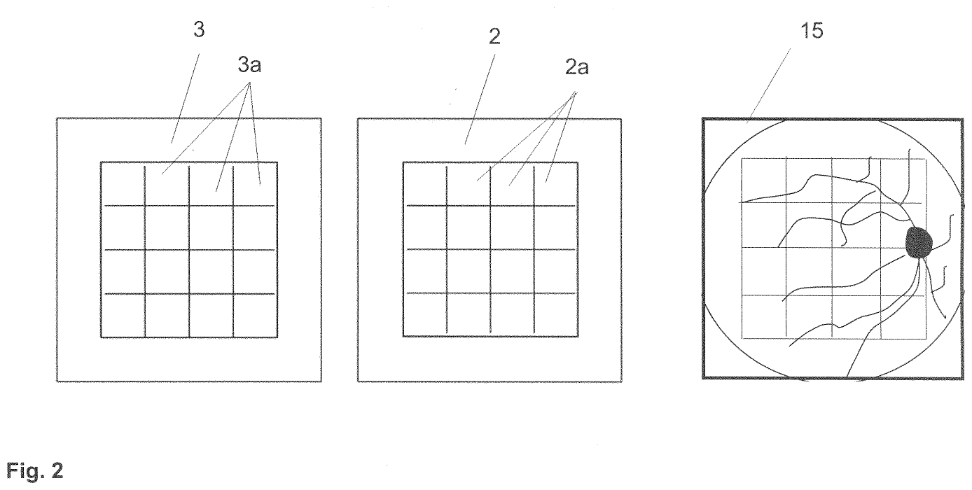

FIG. 2 shows in the center view that the DMD chip 2 has a surface which is divided into a specific number of simulated segments 2a. FIG. 2 shows in the left view that the detector 3 also has a surface which is also divided into a specific number of segments 3a. The segments 2a of the DMD chip 2 each correspond optically with the segments 3a of the detector 3 in order to generate the image 15, which is shown on the right in FIG. 2.

All segments 2a of the DMD chip 2 are equal in size and all segments 3a of the detector 3 are equal in size. The segments 2a of the DMD chip 2 optically correspond to the segments 3a of the detector 3.

Each segment 2a of the DMD chip 2 is assigned precisely one segment 3a of the detector 3, so that the DMD chip 2 can be imaged or projected on the sample side on the surface of the retina 12 of the sample 13 to be examined and on the detector side on the surface of the detector 3. As a result, the retina 12 is imaged on the DMD chip 2 and on the detector 3.

The illumination apparatus comprises the illumination unit 1, wherein the light coming from the illumination unit 1 can be guided onto the DMD chip 2. The DMD chip 2 is thus used to illuminate the sample 13. FIG. 1 specifically shows that the light coming from the illumination unit 1 is guided by means of a TIR prism 5 onto the DMD chip 2 and is guided from there in the direction of the sample 13. A beam splitter 4 is arranged between the illumination unit 1 and the TIR prism 5, by which a light beam reflected from the sample 13 can be deflected onto the detector 3. A light beam originating from the illumination unit 1 can be guided through a first lens 6 and a second lens 7 before the light beam is incident on the TIR prism 5 and from there on the DMD chip 2. The beam splitter 4 is arranged between the first lens 6 and the second lens 7.

Outgoing light beams from the DMD chip 2 to the sample 13 are guided through a third lens 8, a fourth lens 9, and a fifth lens 10 before they strike the sample 13. The sample 13 is the human eye here, the lens 20 of which is used to deflect or focus light beams or light beam bundles on the retina 12.

A detector lens 11 or sixth lens 11 is arranged between the beam splitter 4 and the detector 3. An individual mirror of the DMD chip 2 functions as a pinhole aperture which limits the amount of light.

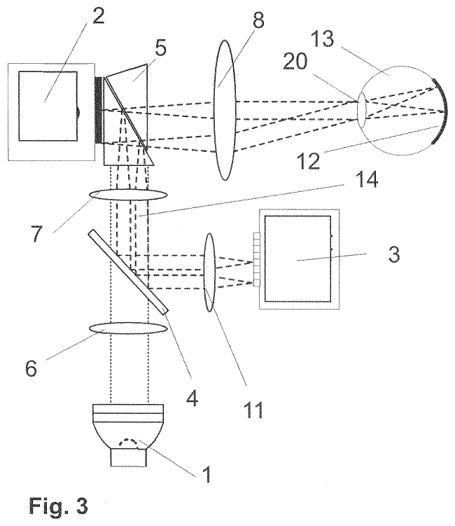

FIG. 3 shows a further system, which is constructed analogously to the system according to FIG. 1, wherein two lenses of the lens arrangement are omitted, however.

In both devices of FIGS. 1 and 3, the detector 3, the DMD chip 2, and the retina 12 of an eye 13 are each optically imaged on one another.

The DMD chip 2, which consists of many separately actuatable mirrors, is imaged via the mentioned optical components on the retina 12. Each individual mirror can correspond to one pixel of a generated image 15. The individual mirrors have very small surfaces. The diagonal of such a surface is approximately 10 μm.

The mirrors can be controlled and moved at a very high speed, and typical movement frequencies are 30 kHz or greater.

The detection takes place in that coupling occurs into the illumination path 14 via the beam splitter 4 of the light-sensitive detector 3 in order to detect light returning from the sample 13, namely the eye. The DMD chip 2 is imaged via optical components on the surface of the detector 3.

To generate the image 15, the individual mirrors are individually switched through at high speed and scan the ocular fundus point by point, similarly as in conventional laser scanning systems.

The light is thus conducted from a mirror via an optical path to the ocular fundus. The light reflected or scattered on the ocular fundus returns on the same optical path and is guided via the currently active individual mirror and the beam splitter 4 onto the detector 3.

The intensity at the detector 3 is maximal when the light beam is reflected in spot form out of the focal plane. Light from outside the focal plane is not reflected via the active mirror for the most part. A confocal principle is thus implemented. A measured value on the detector 3 is assigned to each individual point, from which a two-dimensional image 15 can be assembled.

FIG. 2 shows that instead of an individual light-sensitive detector, a surface detector having multiple segments or channels 3a, which can be read out individually, is used here as the detector 3. The DMD chip 2 is accordingly also divided into segments 2a in the same manner.

The so-called pixel clock or the speed of the image generation can be greatly increased by segments which operate in parallel.

Due to the spatial separation (or division and segments which scan in parallel) of the active mirrors of the DMD chip 2 or the pixels, the confocal principle is maintained, in contrast to a CCD or CMOS camera.

The illumination unit 1 is used for the homogeneous planar illumination of the optically active DMD chip 2. An aperture diaphragm is imaged on the DMD chip 2. The beam guiding takes place in FIG. 1 and FIG. 3 via a TIR prism 5, but can also be implemented without one. Since the amount of light is distributed on the entire surface of the DMD chip 2 or the equivalent surface of the retina 12, a high light power is necessary. This can be implemented, for example, using an IR LED.

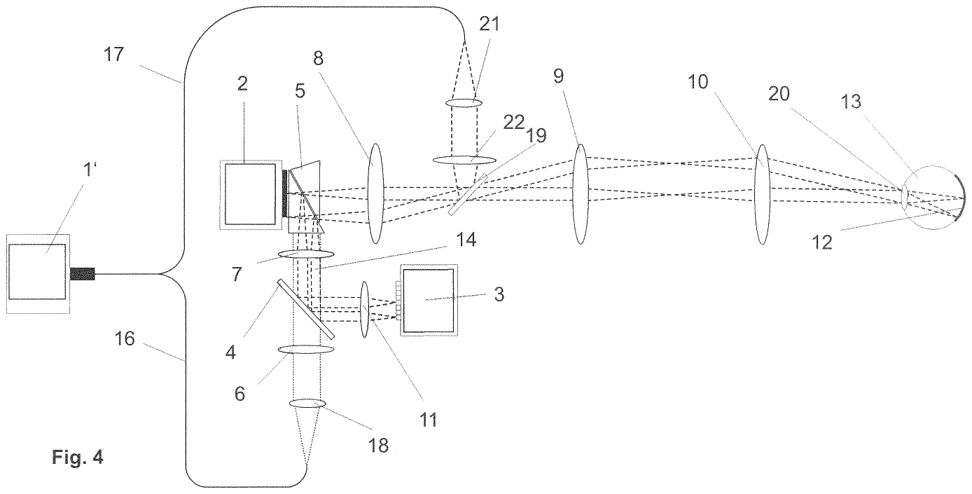

FIG. 4 shows an arrangement which essentially comprises a system according to FIG. 1. The illumination apparatus comprises an illumination unit 1′or swept source OCT illumination unit 1′, the emitted light of which can be split into a sample arm 16 and a reference arm 17, wherein the light signals from sample arm 16 and reference arm 17 can be overlaid at the detector 3 such that they interfere, and wherein OCT images can be generated from the interference signals by means of an evaluation apparatus.

The light of the sample arm 16 can be guided via a feed lens 18 via the first lens 6 via the illumination path 14 to the DMD chip 2, wherein the light of the reference arm 17 can be guided via a deflection mirror or reference light beam splitter 19 via the excitation lens 8 to the DMD chip 2.

The light of the sample arm 16 passes from the DMD chip 2 to the sample 13. From the sample 13, light reflected from the latter is incident again on the DMD chip 2 and from there via the TIR prism 5 and the beam splitter 4 to the detector 3.

The light of the reference arm 17 is conducted by means of a decoupling lens 21 and a coupling lens 22 onto the reference light beam splitter 19 and from there through the excitation lens 8 in the direction toward the DMD chip 2 and from there via the TIR prism 5 and the beam splitter 4 to the detector 3.

At the detector 3, the light beams of the sample arm 13 and the reference arm 17 interfere. OCT images can be generated by evaluating the interference.

FIG. 4 shows in this respect an expansion of the system described here for use in optical coherence tomography (OCT).

FIG. 4 shows the optical structure for OCT pictures. The illumination unit 1′is designed here as a swept source light source and is divided into two arms, namely the sample arm 16 and the reference arm 17. The light signals from sample arm 16 and reference arm 17 are superimposed again and interfere at the detector 3. The length of the two arms 16, 17 has to be adapted to one another.

A frequency of, for example, 32 kHz could be operated using a swept source light source. Therefore, an effective A scanning rate of 2 MHz is implementable using 64 channels. This corresponds to a factor which is eight times faster than at a frequency of 250 kHz. 2D laser scanning, OCT, and a display for patients could run via the same DMD chip. A binocular solution is also conceivable due to the compact dimensions.

### LIST OF REFERENCE NUMERALS

1,1′illumination unit
2 DMD chip
3 detector
4 beam splitter
5 TIR prism
6 first lens
7 second lens
8 third lens
9 fourth lens
10 fifth lens
11 detector lens
12 retina
13 sample
14 illumination path
15 image
16 sample arm
17 reference arm
18 feed lens
19 reference light beam splitter
20 lens of the human eye
21 decoupling lens
22 coupling lens

1. A system for carrying out a confocal ophthalmoscopy, comprising an illumination apparatus for illuminating a sample, a lens arrangement having multiple lenses for guiding light beams from the illumination apparatus to the sample and from this back to a detector,
wherein the illumination apparatus comprises a DMD chip, by which a light beam can be guided to the sample and by which a light beam reflected to it by the sample can be guided to the detector.

2. The system as claimed in claim 1, wherein a light beam or light beam bundle can be guided on the same optical path from the DMD chip to the sample and from this back to the DMD chip as a reflected light beam or reflected light beam bundle, so that the same individual mirror of the DMD chip is usable for illuminating the sample using the light beam or light beam bundle incident thereon and for receiving the light beam or light beam bundle reflected from the sample.

3. The system as claimed in claim 2, wherein the detector is designed as a surface detector and/or MPPC array detector.

4. The system as claimed in claim 3, wherein the DMD chip comprises a surface which is divided into a specific number of segments, wherein the detector comprises a surface which is also divided into a specific number of segments and wherein the segments of the DMD chip each optically correspond with the segments of the detector to generate an image.

5. The system as claimed in claim 4, wherein all segments of the DMD chip are equal in size, wherein all segments of the detector are equal in size, and/or in that the segments of the DMD chip are equal in size to the segments of the detector.

6. The system as claimed in claim 4, wherein each segment of the DMD chip is assigned precisely one segment of the detector, so that the DMD chip can be imaged or projected on the sample side on the surface of the sample to be examined and on the detector side on the surface of the detector.

7. The system as claimed in claim 3, wherein that the illumination apparatus comprises an illumination unit, wherein the light coming from the illumination unit can be guided onto the DMD chip or wherein the light coming from the illumination unit can be guided by means of a TIR prism onto the DMD chip

8. The system as claimed in claim 7, wherein a beam splitter, by which a reflected light beam can be deflected onto the detector, is arranged between the illumination unit and the DMD chip and/or a TIR prism.

9. The system as claimed in claim 7, wherein an outgoing light beam from the illumination unit can be guided through a first lens and a second lens before the light beam strikes the TIR prism and/or the DMD chip.

10. The system as claimed in claim 9, wherein a beam splitter is arranged between a first lens and a second lens.

11. The system as claimed in claim 9, wherein outgoing light beams from the DMD chip toward the sample can be guided through a third lens alone or through a third lens, a fourth lens, and a fifth lens before they strike the sample.

12. The system as claimed in claim 11, wherein a detector lens or sixth lens is arranged between a beam splitter and the detector.

13. The system as claimed in claim 1, wherein an individual mirror of the DMD chip functions as a pinhole aperture.

14. An arrangement comprising a system as claimed in claim 3, wherein the illumination apparatus comprises an illumination unit or swept source OCT illumination unit, the emitted light of which can be split into a sample arm and a reference arm, wherein the light signals from sample arm and reference arm can be overlaid at the detector such that they interfere, and wherein OCT images can be generated from the interference signals by means of an evaluation apparatus.

15. The arrangement as claimed in claim 14, wherein the light of the sample arm can be guided via a feed lens to the DMD chip, wherein the light of the reference arm can be guided via a deflection mirror or reference light beam splitter to the DMD chip.
